(19) United States
(12) Patent Application Publication
(10) Pub. No.: US 2016/0089341 A1
(43) Pub. Date: Mar. 31, 2016

(54) METHODS FOR TREATING AND PREVENTING STATUS EPILEPTICUS AND ORGANOPHOSPHATE POISONING

(71) Applicant: University of Iowa Research Foundation, Iowa City, IA (US)

(72) Inventor: Max T. Baker, Iowa City, IA (US)

(21) Appl. No.: 14/865,656

(22) Filed: Sep. 25, 2015

Related U.S. Application Data
(60) Provisional application No. 62/055,284, filed on Sep. 25, 2014.

(51) Int. Cl. A61K 31/05; A61K 45/06

(52) U.S. Cl. CPC A61K 31/05; A61K 45/06

(57) ABSTRACT

Disclosed are compounds, compositions and methods for stopping seizures, including status epilepticus, and are useful for the treatment and prevention of seizure disorders.

### CROSS-REFERENCE TO RELATED APPLICATIONS

This application claims the benefit of priority U.S. Provisional Application Ser. No. 62/055,284 filed 25 Sep. 2014; entitled METHODS FOR TREATING AND PREVENTING STATUS EPILEPTICUS AND ORGANOPHOSPHATE POISONING; the disclosure of which is hereby incorporated by reference in its entirety.

### BACKGROUND OF THE INVENTION

Seizures involve an excessive firing of neurons in the central nervous system. Such neuronal activity causes cognitive disturbances. There are many types of seizures. Seizures range from those that have mild outward manifestations such as tics or mental pauses, to those that have severe convulsant activities such as tonic-clonic convulsions.

It is well known that seizures are difficult to treat in human and animal subjects. Experience with human patients reveals that no anticonvulsant medication is effective for all seizure types. Furthermore, seizures in individual patients are often treated with more than one anticonvulsant. Animal models show that individual anticonvulsant compounds are selective for seizure types. For example, the commercial anticonvulsant levetiracetam is effective for halting seizures in the mouse 6 Hz Psychomotor model (6 Hz Test), but does not have anticonvulsant activity in the Maximal Electroshock Model (MES). Phenytoin has good anticonvulsant activity in the MES Test, but poor activity in the 6 Hz Test. Neither compound is effective in halting seizures in the Pilocarpine Model test.

Certain types of seizures such as status epilepticus are especially difficult to treat. Status epilepticus is a life-threatening prolonged seizure or series of severe seizures. Some patients may have an unexplained or trauma- or illness-induced status epilepticus. Seizures resulting from deliberate or accidental organophosphate exposure are also a form of status epilepticus and are also very difficult to stop. Organophosphates include the highly lethal compounds known as nerve gases which include tabun, sarin, and soman. They also include a number of lesser toxic, but still seizure-inducing organophosphates used as insecticides (such as parathion).

Most anticonvulsant drugs are poorly effective or ineffective in treating status epilepticus that occurs in humans and in organophosphate-induced seizures in experimental animals. Benzodiazepines, including lorazepam or midazolam, and barbiturates such as phenobarbital or pentobarbital and the alkyl phenol propofol, are drugs used in intractable seizures. However, they are not always successful. They are also not effective in halting seizures in animal models of status epilepticus. Experimental animal models for status epilepticus include those that induce seizures with pilocarpine or soman. Benzodiazepines, for example, are effective when seizures induced by pilocarpine or soman initially start but become ineffective 20-40 min after seizure onset. Seizures at delayed periods after onset are termed “benzodiazepine-resistant”.

Severe seizures arising from conditions such as status epilepticus, trauma and organophosphate poisonings are difficult to halt using current anticonvulsant or antiseizure medications such as benzodiazepines and barbiturates. New treatments for seizures and organophosphate poisoning are needed.

### SUMMARY OF THE INVENTION

Disclosed are compounds and compositions that are effective in stopping seizures especially status epilepticus and are useful for the treatment and prevention of seizure disorders.

In one aspect, the present disclosure provides a method for treating or preventing seizures or seizure disorders, including status epilepticus. The method comprises the step of administering to a subject in need thereof an effective amount of 2,6-di-sec-butylphenol (which can be racemic 2,6-di-sec-butylphenol).

In another aspect, the present disclosure provides a method for treating or preventing organophosphate poisoning or organophosphate-induced seizures. The method comprises the step of administering to a subject in need thereof an effective amount of 2,6-di-sec-butylphenol.

In certain embodiments, 2,6-di-sec-butylphenol is administered orally. In certain embodiments, 2,6-di-sec-butylphenol is administered by inhalation. In certain embodiments, 2,6-di-sec-butylphenol is administered intranasally. In certain embodiments, 2,6-di-sec-butylphenol is administered intramuscularly. In certain embodiments, 2,6-di-sec-butylphenol is administered orally. In certain embodiments, 2,6-di-sec-butylphenol is administered subcutaneously.

The present disclosure also provides the following:

The compound 2,6-di-sec-butylphenol, or a pharmaceutical composition thereof, for the treatment of central nervous system (CNS) disorders.

The compound 2,6-di-sec-butylphenol, or a pharmaceutical composition thereof, for the treatment of seizures.

The compound 2,6-di-sec-butylphenol, or a pharmaceutical composition thereof, for the treatment of status epilepticus.

The compound 2,6-di-sec-butylphenol, or a pharmaceutical composition thereof, for the treatment of organophosphate poisoning or organophosphate-induced seizures.

The compound 2,6-di-sec-butylphenol, or a pharmaceutical composition thereof,

The compound 2,6-di-sec-butylphenol (R,R; R,S; and/or S,S), in which one or more hydrogen atoms is replaced by one or more deuterium atoms.

The compound 2,6-di-sec-butylphenol (R,R; R,S; and/or S,S), in which one or more hydrogen atoms is replaced by one or more deuterium atoms, for the treatment of seizures.

The compound 2,6-di-sec-butylphenol (R,R; R,S; and/or S,S), in which one or more hydrogen atoms is replaced by one or more deuterium atoms, for the treatment of status epilepticus.

The compound 2,6-di-sec-butylphenol (R,R; R,S; and/or S,S), in which one or more hydrogen atoms is replaced by one or more deuterium atoms, for the treatment of organophosphate-induced seizures.

A pharmaceutical composition comprising 2,6-di-sec-butylphenol and an anticonvulsant, such as an anticonvulsant selected from the group consisting of valpromide (VPD), valnoctamide (VCD), or sec-butyl-propylacetamide (SPD).

A pharmaceutical composition comprising 2,6-di-sec-butylphenol and an anticonvulsant, such as an anticonvulsant selected from the group consisting of valpromide (VPD), valnoctamide (VCD), and sec-butyl-propylacetamide (SPD) for the treatment of CNS disorders including seizures.

A pharmaceutical composition comprising and an anticonvulsant, such as an anticonvulsant selected from the group consisting of valpromide (VPD), valnoctamide (VCD), and sec-butyl-propylacetamide (SPD) for the treatment of status epilepticus.

A pharmaceutical composition comprising 2,6-di-sec-butylphenol and an anticonvulsant, such as an anticonvulsant selected from the group consisting of valpromide (VPD), valnoctamide (VCD), and sec-butyl-propylacetamide (SPD) for the treatment of organophosphate-induced seizures.

A pharmaceutical composition comprising R,R; R,S; and/or S,S 2,6-di-sec-butylphenol and one of more of valpromide (VPD), valnoctamide (VCD), and sec-butyl-propylacetamide (SPD).

A pharmaceutical composition comprising R,R; R,S; and/or S,S 2,6-di-sec-butylphenol and an anticonvulsant, such as an anticonvulsant selected from the group consisting of valpromide (VPD), valnoctamide (VCD), and sec-butyl-propylacetamide (SPD) for the treatment of seizures.

A pharmaceutical composition comprising R,R; R,S; and/or S,S 2,6-di-sec-butylphenol and one or more of the stereoisomers of valnoctamide or sec-butyl-propylacetamide.

A pharmaceutical composition comprising R,R; R,S; and/or S,S 2,6-di-sec-butylphenol and one or more of the stereoisomers of valnoctamide or sec-butyl-propylacetamide for the treatment of seizures.

A pharmaceutical composition comprising R,R; R,S; and/or S,S 2,6-di-sec-butylphenol and one or more of the stereoisomers of valnoctamide or sec-butyl-propylacetamide for the treatment of status-epilepticus.

A pharmaceutical composition comprising R,R; R,S; and/or S,S 2,6-di-sec-butylphenol and one or more of the stereoisomers of valnoctamide or sec-butyl-propylacetamide for the treatment of organophosphate-induced seizures.

Other embodiments, features, and advantages of the present disclosure are described hereinbelow.

### DESCRIPTION OF THE FIGURES

FIG. 1 shows seizure EEG activity resulting from 2,6-di-sec-butylphenol administration to a rat having seizures induced by pilocarpine.

FIG. 2 shows seizure EEG activity resulting from propofol administration to a rat having seizures induced by pilocarpine.

### DETAILED DESCRIPTION OF THE INVENTION

The Applicant has discovered that 2,6-di-sec-butylphenol, a racemic compound, has anticonvulsant activity. The Applicant has further discovered that 2,6-di-sec-butylphenol has anticonvulsant properties in status epilepticus and organophosphate poisoning animal models upon delayed administration.

### Terms and Definitions

The term “seizure”, as used herein, refers to excessive neuronal firing in the brain. Seizures can usually be detected by electroencephalogram (EEG) monitoring. “Convulsions” refers to the outward motor movements resulting from a seizure. Although not common, a subject can have a seizure without convulsions.

The term “status epilepticus” or “SE”, as used herein, refers to a life-threatening condition in which the brain is in a state of persistent seizure. SE is defined as one continuous, unremitting seizure lasting longer than five minutes, or recurrent seizures without regaining consciousness between seizures for greater than five minutes. The term “subject” or “patient,” as used herein, refers to an animal, preferably a mammal, including a dog, cat, monkey, horse, cow, sheep, or pig, and, in certain preferred embodiments, a human.

The term “organophosphate”, as used herein, refers to an organic compound containing one or more phosphate or phosphinate ester groups (e.g., an organic ester of phosphoric or phosphinic acid). The term “organophosphate” also includes sulfur analogs of organophosphates unless otherwise stated. Organophosphates are often acetylcholinesterase inhibitors, which are neurotoxic and used as pesticides or nerve agents. Examples of organophosphates include pesticides such as parathion, malathion, methyl parathion, chlorpyrifos, diazinon, dichlorvos, phosmet, fenitrothion, tetrachlorvinphos, azamethiphos, and azinphos methyl, and nerve agents such as tabun, sarin, VX, and soman.

The term “effective amount” includes an amount effective, at dosages and for periods of time necessary, to achieve the desired result, e.g., sufficient to treat or prevent seizures or organophosphate poisoning in the subject. An effective amount of compound of the present disclosure may vary according to factors such as the health, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the compound are outweighed by the therapeutically beneficial effects.

A therapeutically effective amount of a compound of the present disclosure (i.e., an effective dosage) may range from about 0.5 to 500 mg/kg body weight, preferably about 1 to 400 mg/kg body weight, more preferably about 5 to about 300 mg/kg body weight, and even more preferably about 10 to 200 mg/kg, 20 to 100 mg/kg body weight.

The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound of the present disclosure can include a single treatment or, in certain embodiments, can include a series of treatments.

2,6-Di-sec-butylphenol can be administered before or after the onset of seizure activity. In general, administration of 2,6-di-sec-butylphenol is more effective for the prevention of seizures when the administration occurs before or at the onset of seizure activity. However, as discuss in the Examples, infra, it has surprisingly been found that administration of 2,6-di-sec-butylphenol at least 10, 20 or 30 minutes after the onset of seizures is effective to inhibit seizure activity, in status epilepticus. Thus, in certain embodiments of the methods of the present disclosure, 2,6-di-sec-butylphenol is administered before or at the onset of seizure activity. In other embodiments, 2,6-di-sec-butylphenol is administered at least 10, 20, 30, or more minutes after the onset of seizure activity.

In certain embodiments, 2,6-di-sec-butylphenol is administered together with one or more additional therapeutic agents. Examples of additional therapeutic agents include anti-seizure medications such as benzodiazepines (including clonazepam, diazepam, and lorazepam), carbamazepine, ethosuximide, felbamate, tiagabine, levetiracetam, lamotrigine, pregabalin, gabapentin, phenytoin and topirimate; anticonvulsants such as valpromide (VPD), valnoctamide (VCD), and sec-butyl-propylacetamide (SPD); treatments for organophosphate poisoning such as anticholinergic agents, e.g., atropine, scopolamine, fesoterodine, tolterodine, oxybutynin, and the like) or oximes such as pralidoxime. The type and amount of the additional therapeutic agent(s) to be administered will depend upon the condition of the patient.

The present disclosure also provides pharmaceutical compositions. The pharmaceutical compositions include an effective amount of a compound of the disclosure (i.e., 2-6-di-secbutylphenol, as a racemate or a mixture of two or more stereoisomers) and a pharmaceutically acceptable carrier. In certain embodiments, a pharmaceutical composition of the present disclosure further includes one or more additional therapeutic agents as described above.

In certain embodiments, these pharmaceutical compositions are suitable for topical or oral administration to a subject. In other embodiments, as described in detail below, the pharmaceutical compositions of the present disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound.

The phrase “pharmaceutically acceptable” refers to those compounds of the present disclosure, compositions containing such compounds, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

In certain embodiments, the pharmaceutically acceptable carrier can be a formulation vehicles similar to those used for propofol, e.g., emulsions, polymeric micelles, cyclodextrins, and the like.

The phrase “pharmaceutically-acceptable carrier” includes pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Compositions containing a compound(s) of the present disclosure include those suitable for oral, nasal, topical (including buccal and sublingual), rectal, vaginal, aerosol and/or parenteral administration. The compositions may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred per cent, this amount will range from about 1 percent to about ninety-nine percent of active ingredient, preferably from about 5 percent to about 70 percent, most preferably from about 0.5% percent to about 5% percent for parenteral administration.

Methods of preparing these compositions include the step of bringing into association a compound(s) of the present disclosure with the carrier and, optionally, one or more accessory ingredients. In general, the formulations are prepared by uniformly and intimately bringing into association a compound of the present disclosure with liquid carriers, or finely divided solid carriers, or both, and then, if necessary, shaping the product.

Compositions of the present disclosure suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound(s) of the present disclosure as an active ingredient. A compound may also be administered as a bolus, electuary or paste.

In solid dosage forms of the present disclosure for oral administration (capsules, tablets, pills, dragees, powders, granules and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient moistened with an inert liquid diluent.

The tablets, and other solid dosage forms of the pharmaceutical compositions of the present disclosure, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compound(s) of the present disclosure include pharmaceutically-acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

In addition to inert diluents, the oral compositions can include adjuvants such as wetting agents, emulsifying and suspending agents, sweetening, flavoring, coloring, perfuming and preservative agents.

Suspensions, in addition to the active compound(s) of the present disclosure may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Pharmaceutical compositions of the present disclosure for rectal or vaginal administration may be presented as a suppository, which may be prepared by mixing one or more compound(s) of the present disclosure with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent.

Compositions of the present disclosure which are suitable for vaginal administration also include pessaries, tampons, creams, gels, pastes, foams or spray formulations containing such carriers as are known in the art to be appropriate.

Dosage forms for the topical or transdermal administration of a compound(s) of the present disclosure include powders, sprays, ointments, pastes, creams, lotions, gels, solutions, patches and inhalants. The active compound(s) of the present disclosure may be mixed under sterile conditions with a pharmaceutically-acceptable carrier, and with any preservatives, buffers, or propellants which may be required.

The ointments, pastes, creams and gels may contain, in addition to compound(s) of the present disclosure, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound(s) of the present disclosure, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

The compound(s) of the present disclosure can be alternatively administered by aerosol. This is accomplished by preparing an aqueous aerosol, liposomal preparation or solid particles containing the compound. A nonaqueous (e.g., fluorocarbon propellant) suspension could be used. Sonic nebulizers are preferred because they minimize exposing the agent to shear, which can result in degradation of the compound.

Ordinarily, an aqueous aerosol is made by formulating an aqueous solution or suspension of the agent together with conventional pharmaceutically-acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches have the added advantage of providing controlled delivery of a compound(s) of the present disclosure to the body. Such dosage forms can be made by dissolving or dispersing the agent in the proper medium. Absorption enhancers can also be used to increase the flux of the active ingredient across the skin. The rate of such flux can be controlled by either providing a rate controlling membrane or dispersing the active ingredient in a polymer matrix or gel.

Ophthalmic formulations, eye ointments, powders, solutions and the like, are also contemplated as being within the scope of the present disclosure.

Pharmaceutical compositions of the present disclosure suitable for parenteral administration comprise one or more compound(s) of the present disclosure in combination with one or more pharmaceutically-acceptable sterile isotonic aqueous or nonaqueous solutions, dispersions, suspensions or emulsions (including oil-in-water emulsions), polymeric micelles, cyclodextrin complexes, liposomes, protein nanoparticle formulations, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the present disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of compound(s) of the present disclosure in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compound(s) of the present disclosure are administered as pharmaceuticals, to humans and animals, they can be given per se or as a pharmaceutical composition containing, for example, 0.1 to 99.5% (more preferably, 0.5 to 90%) of active ingredient in combination with a pharmaceutically-acceptable carrier.

Regardless of the route of administration selected, the compound(s) of the present disclosure, which may be used in a suitable hydrated form, and/or the pharmaceutical compositions of the present disclosure, are formulated into pharmaceutically-acceptable dosage forms by conventional methods known to those of skill in the art.

The term “racemic”, as used herein, refers to a mixture of enantiomers of a chiral compound. As used herein, the term “racemic 2,6-di-sec-butylphenol”, unless specifically stated otherwise, includes a mixture of the R,R; S,S, and R,S (meso) stereoisomers.

Disclosed are compounds and compositions that are effective in stopping seizures especially status epilepticus and are useful for the treatment and prevention of seizure disorders.

In one aspect, the disclosure provides a method for treating or preventing seizures or seizure disorders, including status epilepticus. The method comprises the step of administering to a subject in need thereof an effective amount of 2,6-di-sec-butylphenol (which can be racemic 2,6-di-sec-butylphenol).

In another aspect, the disclosure provides a method for treating or preventing organophosphate poisoning or organophosphate-induced seizures. The method comprises the step of administering to a subject in need thereof an effective amount of 2,6-di-sec-butylphenol.

In certain embodiments, 2,6-di-sec-butylphenol is administered orally. In certain embodiments, 2,6-di-sec-butylphenol is administered by inhalation. In certain embodiments, 2,6-di-sec-butylphenol is administered intranasally. In certain embodiments, 2,6-di-sec-butylphenol is administered intramuscularly. In certain embodiments, 2,6-di-sec-butylphenol is administered orally. In certain embodiments, 2,6-di-sec-butylphenol is administered subcutaneously.

The disclosure also provides the following:

The compound 2,6-di-sec-butylphenol, or a pharmaceutical composition thereof, for the treatment of central nervous system (CNS) disorders.

The compound 2,6-di-sec-butylphenol, or a pharmaceutical composition thereof, for the treatment of seizures.

The compound 2,6-di-sec-butylphenol, or a pharmaceutical composition thereof, for the treatment of status epilepticus.

The compound 2,6-di-sec-butylphenol, or a pharmaceutical composition thereof, for the treatment of organophosphate poisoning or organophosphate-induced seizures.

The compound 2,6-di-sec-butylphenol, or a pharmaceutical composition thereof.

The compound 2,6-di-sec-butylphenol (R,R; R,S; and/or S,S), in which one or more hydrogen atoms is replaced by one or more deuterium atoms.

The compound 2,6-di-sec-butylphenol (R,R; R,S; and/or S,S), in which one or more hydrogen atoms is replaced by one or more deuterium atoms, for the treatment of seizures.

The compound 2,6-di-sec-butylphenol (R,R; R,S; and/or S,S), in which one or more hydrogen atoms is replaced by one or more deuterium atoms, for the treatment of status epilepticus.

The compound 2,6-di-sec-butylphenol (R,R; R,S; and/or S,S), in which one or more hydrogen atoms is replaced by one or more deuterium atoms, for the treatment of organophosphate-induced seizures.

A pharmaceutical composition comprising 2,6-di-sec-butylphenol and an anticonvulsant, such as an anticonvulsant selected from the group consisting of valpromide (VPD), valnoctamide (VCD), or sec-butyl-propylacetamide (SPD).

A pharmaceutical composition comprising 2,6-di-sec-butylphenol and an anticonvulsant, such as an anticonvulsant selected from the group consisting of valpromide (VPD), valnoctamide (VCD), and sec-butyl-propylacetamide (SPD) for the treatment of CNS disorders including seizures.

A pharmaceutical composition comprising and an anticonvulsant, such as an anticonvulsant selected from the group consisting of valpromide (VPD), valnoctamide (VCD), and sec-butyl-propylacetamide (SPD) for the treatment of status epilepticus.

A pharmaceutical composition comprising 2,6-di-sec-butylphenol and an anticonvulsant, such as an anticonvulsant selected from the group consisting of valpromide (VPD), valnoctamide (VCD), and sec-butyl-propylacetamide (SPD) for the treatment of organophosphate-induced seizures.

A pharmaceutical composition comprising R,R; R,S; and/or S,S 2,6-di-sec-butylphenol and one of more of valpromide (VPD), valnoctamide (VCD), and sec-butyl-propylacetamide (SPD).

A pharmaceutical composition comprising R,R; R,S; and/or S,S 2,6-di-sec-butylphenol and an anticonvulsant, such as an anticonvulsant selected from the group consisting of valpromide (VPD), valnoctamide (VCD), and sec-butyl-propylacetamide (SPD) for the treatment of seizures.

A pharmaceutical composition comprising R,R; R,S; and/or S,S 2,6-di-sec-butylphenol and one or more of the stereoisomers of valnoctamide or sec-butyl-propylacetamide.

A pharmaceutical composition comprising R,R; R,S; and/or S,S 2,6-di-sec-butylphenol and one or more of the stereoisomers of valnoctamide or sec-butyl-propylacetamide for the treatment of seizures.

A pharmaceutical composition comprising R,R; R,S; and/or S,S 2,6-di-sec-butylphenol and one or more of the stereoisomers of valnoctamide or sec-butyl-propylacetamide for the treatment of status-epilepticus.

A pharmaceutical composition comprising R,R; R,S; and/or S,S 2,6-di-sec-butylphenol and one or more of the stereoisomers of valnoctamide or sec-butyl-propylacetamide for the treatment of organophosphate-induced seizures.

The present disclosure also provides prodrugs of 2,6-di-sec-butylphenol, including prodrugs of racemic 2,6-di-sec-butylphenol; prodrugs of R,R-2,6-di-sec-butylphenol; prodrugs of S,S-2,6-di-sec-butylphenol; and prodrugs of R,S-2,6-di-sec-butylphenol. The present disclosure also provides compositions and methods of treatment using the prodrugs of the disclosure. A prodrug is a compound that is metabolized, cleaved, or otherwise converted in vivo, after administration to a subject, to 2,6-di-sec-butylphenol. Exemplary prodrugs include phenolic esters, carbamates, phosphates, phosphonates and sulfamates (that is, esters, carbamates, phosphates, phosphonates or sulfamates of the phenolic oxygen atom of 2,6-di-sec-butylphenol).

### EXAMPLES

### Example 1

2,6-di-sec-butylphenol was tested for its ability to treat or prevent seizures in a rat seizure model (the pilocarpine model). In brief, the pilocarpine model is an animal model of status epilepticus (for a review, see, e.g., Curia et al., J Neurosci Methods. Jul. 30, 2008; 172(2-4): 143-157).

2,6-Di-sec-butylphenol (as a mixture of R,S; R,R; and S,S stereoisomers) was obtained from Sigma Aldrich and purified by silica gel chromatography before use. Table 1 shows the comparative effects of 2,6-di-sec-butylphenol with propofol in halting seizures in the rat pilocarpine model. 2,6-Di-sec-butylphenol was found to be completely protective at the time of seizure onset and at 0.5 hours after seizure onset. Propofol on the other hand was not completely protective.

TABLE 1

Dose (i.p.) and time when test
compound was given after onset of
S3-5 seizures. Data are number animals
protected/number of animals treated

| Compound | 65 mg/kg, 0 hour | 65 mg/kg, 0.5 hours |
|---|---|---|
| Propofol | 7/8 | 6/8 |
| 2,6-di-sec-butylphenol | 8/8 | 8/8 |

It can be seen that 2,6-di-sec-butylphenol protected all of the animals treated (8/8) when administered either at the onset of seizures or at 30 minutes after onset of seizures. In contrast, propofol was less effective (7 of 8 animals when administered at the onset of seizures, 6 of 8 when administered at 30 minutes after onset of seizures).

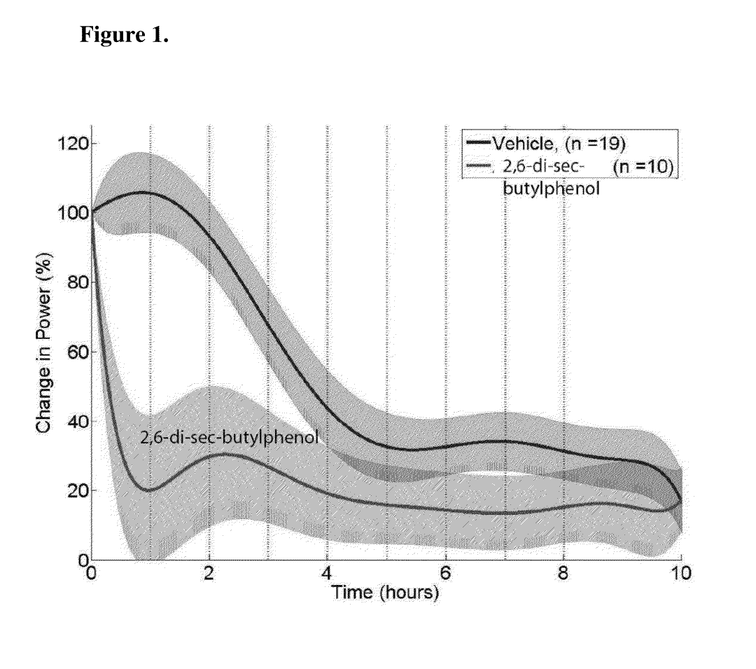

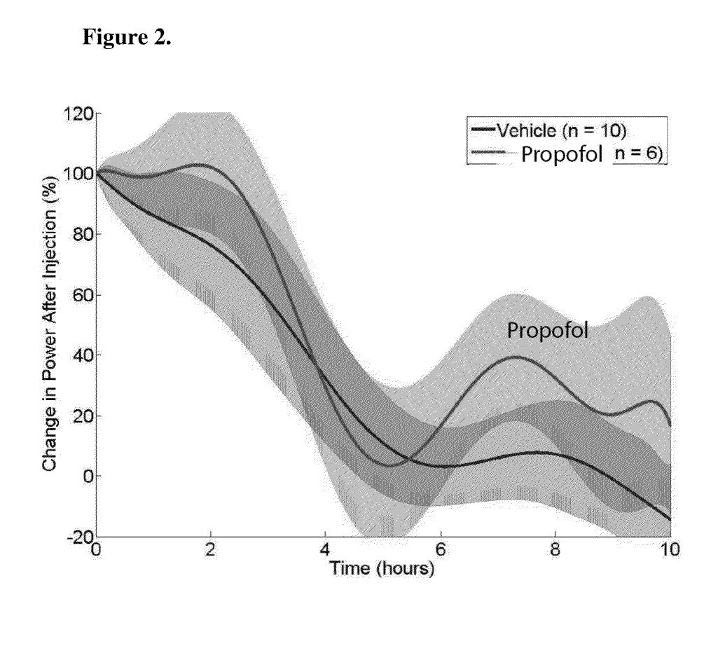

Seizure EEG activity resulting from propofol and 2,6-di-sec-butylphenol administration to rat having seizures induced by pilocarpine is shown in FIG. 1 (2,6-di-sec-butylphenol) and FIG. 2 (propofol). At the same dose of 65 mg/kg i.p. (intraperitoneal), 2,6-di-sec-butylphenol significantly reduced the change in power of EEG activity up to 8 hours. Propofol did not cause a significant reduction in EEG power (FIG. 2).

### Example 2

2,6-Di-sec-butylphenol was tested for its ability to halt seizures induced by the nerve agent soman in the rat. Table 2 shows the effects of 2,6-di-sec-butylphenol at 20 minutes after seizure onset.

TABLE 2

| | Dose (mg/kg, i.p.) | | | | |
|---|---|---|---|---|---|
| | 40 | 50 | 63 | 79 | 100 |
| Number of animals | 0/6 | 1/6 | 1/6 | 3/5 | 2/5 |
| protected/number tested | | | | | |

As shown in Table 2, 2,6-di-sec-butylphenol exhibited significant activity in inhibiting seizures caused by soman when administered 20 minutes after seizure onset.

Those skilled in the art will recognize, or be able to ascertain and implement using no more than routine experimentation, many equivalents of the specific embodiments described herein. Such equivalents are intended to be encompassed by the following claims. Any combination, or combinations, of the embodiments disclosed in the dependent claims are contemplated to be within the scope of the disclosure.

The disclosure of each and every U.S. and foreign patent, published patent application, and publication referred to herein is specifically incorporated by reference herein in its entirety.

What is claimed is:

1. A method for treating or preventing a disease or disorder, the method comprising administering to a subject in need thereof an effective amount of racemic 2,6-di-sec-butylphenol.

2. The method of claim 1, wherein 2,6-di-sec-butylphenol is administered orally, by inhalation, intranasally, intramuscularly, or subcutaneously.

3. The method of claim 1, wherein the disease or disorder is selected from the group consisting of: seizures or a seizure disorder; and organophosphate poisoning or organophosphate-induced seizures,

4. The method of claim 3, wherein the seizure or seizure disorder is status epilepticus.

5. The method of claim 3, wherein when the disease or disorder is organophosphate poisoning or organophosphate-induced seizures the 2,6-di-sec-butylphenol is racemic 2,6-di-sec-butylphenol.

6. A pharmaceutical composition comprising an effective amount of 2,6-di-sec-butylphenol, and a pharmaceutically-acceptable carrier.

7. The pharmaceutical position of claim 6, wherein the 2,6-di-sec-butylphenol is a deuterated 2,6-di-sec-butylphenol in which one or more hydrogen atoms is replaced by one or more deuterium atoms.

8. A composition comprising an effective amount of 2,6-di-sec-butylphenol and an anticonvulsant.

9. The composition of claim 8, further comprising a pharmaceutically-acceptable carrier.

10. The composition of claim 8, wherein the anticonvulsant is selected from the group consisting of valpromide (VPD), valnoctamide (VCD), or sec-butyl-propylacetamide (SPD).

11. A method for the treatment of a disease or disorder, the method comprising administering an effective amount of a composition comprising 2,6-di-sec-butylphenol and an anticonvulsant to a subject in need thereof.

12. The method of claim 11, wherein the composition further comprising a pharmaceutically-acceptable carrier.

13. The method of claim 11, wherein the disease or disorder is selected from the group consisting of: CNS disorders; status epilepticus; and organophosphate poisoning or organophosphate-induced seizures

14. The method of claim 13, wherein the CNS disorder is seizures.

15. The method of claim 11, wherein the anticonvulsant is selected from the group consisting of valpromide (VPD), valnoctamide (VCD), or sec-butyl-propylacetamide (SPD).
